(19) United States
(12) Patent Application Publication
(10) Pub. No.: US 2005/0215589 A1
(43) Pub. Date: Sep. 29, 2005

(54) Inhibitors of 5-HT2A receptor

(76) Inventors: Roger Crossley, Sittingbourne (GB); Terry Ward, Coleville (GB); Malin Berthold, Djursholm (SE)

Correspondence Address: FISH & RICHARDSON PC, P.O. BOX 1022, MINNEAPOLIS, MN, 55440-1022, US

(21) Appl. No.: 10/947,911

(22) Filed: Sep. 23, 2004

Related U.S. Application Data
(63) Continuation of application No. 10/933,921, filed on Sep. 2, 2004, now abandoned.
(60) Provisional application No. 60/505,337, filed on Sep. 23, 2003.
(60) Provisional application No. 60/505,295, filed on Sep. 23, 2003.

(30) Foreign Application Priority Data
Sep. 3, 2003 (SE) 0302369-4

(51) Int. Cl. A61K 31/445; C07D 211/30

(52) U.S. Cl. 514/317; 546/225

(57) ABSTRACT

Compounds of Formula (I): 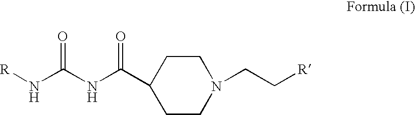
wherein R and R′ are described herein, as are processes for preparing the compounds, pharmaceutical compositions comprising the compounds, and use of the compounds and compositions in the prophylaxis or treatment of a 5-HT_{2A} receptor-related disorder.

### CROSS REFERENCE TO RELATED APPLICATIONS

This application claims priority to U.S. application Ser. No. 10/933,921, filed Sep. 2, 2004; U.S. provisional application Ser. No. 60/505,295, filed Sep. 23, 2003; and to Swedish Patent Application No. 0302369.4, filed Sep. 3, 2003. The prior applications are incorporated herein by reference in their entirety.

### TECHNICAL FIELD

The present invention relates to novel compounds, to pharmaceutical compositions comprising the compounds, to processes for their preparation, as well as to the use of the compounds for the preparation of a medicament against 5-HT_{2A} receptor-related disorders.

### BACKGROUND

Many disorders and conditions of the central nervous system are influenced by the adrenergic, the dopaminergic, and the serotonergic neurotransmitter systems. For example, serotonin (5-HT; 5-hydroxytryptamine) has been implicated in a number of disorders and conditions which originate in the central nervous system.

The HT_{2A} receptor has been implicated as a therapeutic target for the treatment or prevention of abnormalities of the serotonergic system, including psychotic disorders such as schizophrenia (A. Carlsson, N. Waters and M. L. Carlsson, Biol. Psychiatry, 46, 1388 (1999); G. J. Marek and G. K. Aghajanian, Biol. Psychiatry, 44,1118 (1998); E. Sibelle, Z. Sarnyai, D. Benjamin, J. Gal, H. Baker and M. Toth, Mol. Pharmacol., 52, 1056 (1997)). Abnormality of this system has also been implicated in a number of human diseases such as mental depression (Arias B, Gutierrez B, Pintor L, Gasto C, Fananas L, Mol. Psychiatry (2001) 6, 239-242), migraine, epilepsy and obsessive-compulsive disorder (Luisa de Angelis, Current Opinion in Investigational Drugs (2002) 3 (1) 106-112). 5-HT_{2A} antagonists may also be useful in the treatment of sleep disorders such as insomnia and obstructive sleep apnea, anorexia nervosa (Zlegler A, Gorg T, Lancet (1999) 353, 929), cardiovascular conditions such as hypertension, vasospasm, angina, Raynaud's phenomenon and thrombotic illness including stroke, glaucoma (T. Mano et al. and H. Takaneka et al., Investigative Ophthalmology and Visual Science, 1995, vol. 36, pages 719 and 734, respectively) and in the inhibition of platelet aggregation. Evidence also implies that selective 5-HT_{2A} receptor antagonists may also be useful in the treatment of alcohol and cocaine dependence (Maurel S, De Vry J, De Beun R, Schreiber, Pharmacol. Biochem Behav (1999) 89-96; McMahan L R, Cunningham K A, Pharmacol Exp Ther (2001) 297, 357-363).

### SUMMARY

One object of the present invention is a compound of the Formula (I) 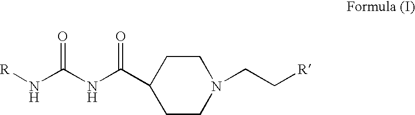
wherein R is either
aryl optionally independently substituted with one or more of C₁₋₆-alkyl, C₁₋₆-alkoxy, halogen, and halo-C₁₋₆-alkyl; or
aryl-C₁₋₆-alkyl optionally independently substituted with one or more of C₁₋₆-alkoxy; or
C₃₋₈-cycloalkyl;
R′ is either
aryl optionally independently substituted with one or more of halogen, C₁₋₆-alkoxy, halo-C₁₋₆-alkyl, and cyano; or
aryloxy optionally independently substituted with one or more of halogen and C₁₋₆-alkoxy; or
heteroaryl optionally independently substituted with one aryl and/or one or more of halogen, C₁₋₆-alkyl, and C₁₋₆-alkoxy, which aryl is optionally independently substituted with one or more of halogen, C₁₋₆-alkyl, and C₁₋₆-alkoxy; and pharmaceutically acceptable salts, hydrates, solvates, geometrical isomers, tautomers, optical isomers, and prodrug forms thereof.

It is preferred that R is selected from
phenyl independently substituted with one or more of methyl, methoxy, ethoxy, fluoro, and trifluoromethyl;
benzyl independently substituted with one or more of methoxy; and
cyclohexyl.

It is especially preferred that R is selected from 2-ethoxyphenyl, 2,4-difluorophenyl, 3-(trifluoromethyl)phenyl, 3,4,5-trimethoxybenzyl, and cyclohexyl.

It is preferred that R′ is selected from
phenyl independently substituted with one or more of fluoro;
phenoxy independently substituted with one or more of methoxy; and
indolyl independently substituted with one phenyl and/or one or more of fluoro, chloro, methyl, and methoxy, which phenyl is optionally independently substituted with one or more of fluoro, chloro, methyl, and methoxy.

It is especially preferred that R′ is selected from 4-fluorophenyl, 2,6-dimethoxyphenoxy, and 2-phenyl-3-indolyl.

Preferred compounds are given in Examples 1-5.

Another object of the present invention is a process for the preparation of a compound as mentioned above, which process comprises the step of
a) reacting an amine RNH₂
wherein R is either
aryl optionally independently substituted with one or more of C₁₋₆-alkyl, C₁₋₆-alkoxy, halogen, and halo-C₁₋₆-alkyl; or
aryl-C₁₋₆-alkyl optionally independently substituted with one or more of C₁₋₆-alkoxy; or
C₃₋₈-cycloalkyl;
with a cyanate, to give a compound of formula R—NH—CO—NH₂,
wherein R is as defined above,
b) alkylation of a compound of Formula (II) 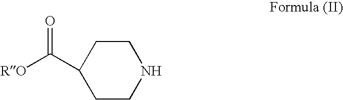
wherein R″ is C₁₋₆-alkyl,
via displacement of a leaving group by reaction of the compound of Formula (II) with an alkylating agent of the Formula R′—CH₂—CH₂-LG,
wherein R′ is either
aryl optionally independently substituted with one or more of halogen, C₁₋₆-alkoxy, halo-C₁₋₆-alkyl, and cyano; or
aryloxy optionally independently substituted with one or more of halogen and C₁₋₆-alkoxy; or
heteroaryl optionally independently substituted with one aryl and/or one or more of halogen, C₁₋₆-alkyl, and C₁₋₆-alkoxy, which aryl is optionally independently substituted with one or more of halogen, C₁₋₆-alkyl, and C₁₋₆-alkoxy; and
LG is a leaving group,
to give a compound of Formula (III) 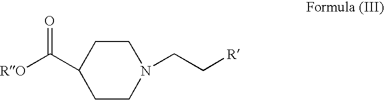
   wherein R′ and R″ are as defined above,
c) reacting the products from steps a) and b) in the presence of a base, such as sodium methoxide or potassium tert-butoxide, to give a compound of Formula (I) 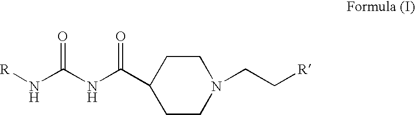
   wherein R and R′ are as defined above.

Another object of the present invention is a compound as mentioned above for use in therapy, especially for use in the prophylaxis or treatment of a 5-HT_{2A} receptor-related disorder.

Another object of the present invention is a pharmaceutical formulation comprising a compound as mentioned above as active ingredient, in combination with a pharmaceutically acceptable diluent or carrier, especially for use in the prophylaxis or treatment of a 5-HT_{2A} receptor-related disorder.

Another object of the present invention is a method for treating a human or animal subject suffering from a 5-HT_{2A} receptor-related disorder. The method can include administering to a subject (e.g., a human or an animal, dog, cat, horse, cow) in need thereof an effective amount of one or more compounds of any of the formulae herein, their salts, or compositions containing the compounds or salts.

The methods delineated herein can also include the step of identifying that the subject is in need of treatment of the 5-HT_{2A} receptor-related disorder. Identifying a subject in need of such treatment can be in the judgment of a subject or a health care professional and can be subjective (e.g., opinion) or objective (e.g., measurable by a test or diagnostic method).

Another object of the present invention is a method for the prophylaxis of a 5-HT_{2A} receptor-related disorder, which comprises administering to a subject in need of such treatment an effective amount of a compound as mentioned above.

Another object of the present invention is a method for modulating 5-HT_{2A} receptor activity, which comprises administering to a subject in need of such treatment an effective amount of a compound as mentioned above.

Another object of the present invention is the use of a compound as mentioned above for the manufacture of a medicament for use in the prophylaxis or treatment of a 5-HT_{2A} receptor-related disorder.

The compounds as mentioned above may be agonists, partial agonists or antagonists for the 5-HT_{2A} receptor.

Examples of 5-HT_{2A} receptor-related disorders are schizophrenia, mental depression, migraine, epilepsy, obsessive-compulsive disorder, sleep disorders such as insomnia and obstructive sleep apnea, anorexia nervosa, cardiovascular conditions such as hypertension, vasospasm, angina, Raynaud's phenomenon and thrombotic illness including stroke, glaucoma, alcohol and cocaine dependence.

The compounds and compositions are useful for treating diseases, including schizophrenia, mental depression, migraine, epilepsy, obsessive-compulsive disorder, sleep disorders such as insomnia and obstructive sleep apnea, anorexia nervosa, cardiovascular conditions such as hypertension, vasospasm, angina, Raynaud's phenomenon and thrombotic illness including stroke, glaucoma, alcohol and cocaine dependence. In one aspect, the invention relates to a method for treating or preventing an aforementioned disease comprising administrating to a subject in need of such treatment an effective amount of a compound or composition delineated herein.

Unless otherwise stated or indicated, the term “C₁₋₆-alkyl” denotes a straight or branched alkyl group having from 1 to 6 carbon atoms. Examples of said lower alkyl include methyl, ethyl, n-propyl, iso-propyl, n-butyl, iso-butyl, sec-butyl, t-butyl and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. “Halo-C₁₋₆-alkyl” means a C₁₋₆-alkyl group substituted with one or more halogen atoms. Likewise, “aryl-C₁₋₆-alkyl” means a C₁₋₆-alkyl group substituted with one or more aryl groups.

Unless otherwise stated or indicated, the term “C₃₋₈-cycloalkyl” denotes a cyclic alkyl group having a ring size from 3 to 8 carbon atoms. Examples of said cycloalkyl include cyclopropyl, cyclobutyl, cyclopentyl, cyclohexyl, methylcyclohexyl, cycloheptyl, and cyclooctyl. For parts of the range “C₃₋₈-cycloalkyl” all subgroups thereof are contemplated such as C₃₋₇-cycloalkyl, C₃₋₆-cycloalkyl, C₃₋₅-cycloalkyl, C₃₋₄-cycloalkyl, C₄₋₈-cycloalkyl, C₄₋₇-cycloalkyl, C₄₋₆-cycloalkyl, C₄₋₅-cycloalkyl, C₅₋₇cylcoalkyl, C₆₋₇-cycloalkyl, etc.

Unless otherwise stated or indicated, the term “C₁₋₆ alkoxy” denotes a straight or branched alkoxy group having from 1 to 6 carbon atoms. Examples of said lower alkoxy include methoxy, ethoxy, n-propoxy, iso-propoxy, n-butoxy, iso-butoxy, sec-butoxy, t-butoxy and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc.

Unless otherwise stated or indicated, the term “halogen” shall mean fluorine, chlorine, bromine or iodine.

Unless otherwise stated or indicated, the term “aryl” refers to a hydrocarbon ring system having at least one aromatic ring. Examples of aryls are phenyl, pentalenyl, indenyl, indanyl, isoindolinyl, chromanyl, naphthyl, fluorenyl, anthryl, phenanthryl and pyrenyl. The aryl rings may optionally be substituted with C₁₋₆-alkyl. Examples of substituted aryl groups are 2-methylphenyl and 3-methylphenyl. Likewise, “aryloxy” refers to an aryl group bonded to an oxygen atom.

The term “heteroaryl” refers to a hydrocarbon ring system having at least one aromatic ring having one or more ring atoms are a heteroatom such as O, N, or S, and the remaining ring atoms are carbon. Examples of heteroaryl groups include furyl, pyrrolyl, thienyl, oxazolyl, isoxazolyl, imidazolyl, thiazolyl, isothiazolyl, pyridinyl, pyrimidinyl, quinazolinyl, indolyl, pyrazolyl, pyridazinyl, quinolinyl, benzofuranyl, dihydrobenzofuranyl, benzodioxolyl, benzodioxinyl, benzothiazolyl, benzothiadiazolyl, and benzotriazolyl groups.

The term “leaving group” refers to a group to be displaced from a molecule during a nucleophilic displacement reaction. Examples of leaving groups are iodide, bromide, chloride, methanesulfonate, hydroxy, methoxy, thiomethoxy, tosyl, or suitable protonated forms thereof (e.g., H₂O, MeOH), especially bromide and methanesulfonate.

The term “alkylating agent” refers to a compound containing one or more alkyl groups which can be added to another compound. Examples of alkylating agents include, but are not limited to, iodomethane, iodoethane, 1-iodopropane, 2-iodopropane, straight- and branched-iodobutane, iodopentane, iodohexane, bromomethane, bomoethane, 1-bromopropane, 2-bromopropane, straight- and branched-bromobutane, bromopentane, bromohexane, allyl bromide, ethyl methanesulfonate, methyl methanesulfonate, and propyl methanesulfonate.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “prodrug forms” means a pharmacologically acceptable derivative, such as an ester or an amide, which derivative is biotransformed in the body to form the active drug. Reference is made to Goodman and Gilman's, The Pharmacological basis of Therapeutics, 8^{th} ed., Mc-Graw-Hill, Int. Ed. 1992, “Biotransformation of Drugs”, p. 13-15.

The following abbreviations have been used:
ACN means acetonitrile,
CHO means Chinese hamster ovary,
DEA means diethylamine,
DEPT means distortion enhancement polarisation transfer,
DMSO means dimethyl sulfoxide,
ELS means electron light scattering,
HPLC means high performance liquid chromatography,
Rt means retention time,
TFA means trifluoroacetic acid,
THF means tetrahydrofuran,
TLC means thin layer chromatography.

All isomeric forms possible (pure enantiomers, diastereomers, tautomers, racemic mixtures and unequal mixtures of two enantiomers) for the compounds delineated are within the scope of the invention. Such compounds can also occur as cis- or trans-, E- or Z-double bond isomer forms. All isomeric forms are contemplated.

The compounds of the formula (I) may be used as such or, where appropriate, as pharmacologically acceptable salts (acid or base addition salts) thereof. The pharmacologically acceptable addition salts mentioned above are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulfuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulfonic acid, toluenesulfonic acid, methanesulfonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

In a further aspect the invention relates to methods of making compounds of any of the formulae herein comprising reacting any one or more of the compounds of the formulae delineated herein, including any processes delineated herein. The compounds of the formula (I) above may be prepared by, or in analogy with, conventional methods.

The processes described above may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g. as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser 's Reagents for Organic Synthesis, John Wiley and Sons (1994); and L. Paquette, ed., Encyclopedia of Reagents for Organic Synthesis, John Wiley and Sons (1995) and subsequent editions thereof.

The necessary starting materials for preparing the compounds of formula (I) are either known or may be prepared in analogy with the preparation of known compounds. The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The details of one or more embodiments of the invention are set forth in the accompanying drawings and the description below. Other features, objects, and advantages of the invention will be apparent from the description and drawings, and from the claims.

### DETAILED DESCRIPTION

In the examples below, all reagents were commercial grade and were used as received without further purification, unless otherwise specified. Commercially available anhydrous solvents were used for reactions conducted under inert atmosphere. Reagent grade solvents were used in all other cases, unless otherwise specified. Column chromatography was performed on Matrex® silica gel 60 (35-70 micron). TLC was carried out using pre-coated silica gel F-254 plates (thickness 0.25 mm). ¹H NMR spectra were recorded on a Bruker Avance250 at 250 MHz. Chemical shifts for ¹H NMR spectra are given in part per million and either tetramethylsilane (0.00 ppm) or residual solvent peaks were used as internal reference. Splitting patterns are designated as follows: s, singlet; d, doublet; t, triplet; q, quartet; p, pentet; m, multiplet; br, broad. Coupling constants are given in Hertz (Hz). Only selected data are reported. The ¹³C NMR spectra were recorded at 62.5 MHz. DEPT experiments were used to help assign ¹³C NMR resonances where necessary. Chemical shifts for ¹³C NMR spectra are expressed in parts per million and residual solvent peaks were used as internal reference. HPLC analyses were performed using a Waters Xterra MS C18 column (100×4.6 mm, 5 μ) eluting with a gradient of 5% ACN in 95% water to 95% ACN in 5% water (0.2% TFA buffer) over 3.5 mins, then 95% ACN in 5% water (0.2% TFA buffer) for a further 2.5 mins at a flow rate of 3 ml/min on a Waters 600E or Gilson system with monitoring at 254 rn. Reverse phase preparative HPLC was carried out using a Xterra MS C18 column (100×19 mm, 5 μm) eluting with a gradient of 5% ACN in 95% water to 95% ACN in 5% water (0.05% DEA) over 12.0 mins, then 95% ACN in 5% water (0.05% DEA) for a further 5.0 mins at a flow rate of 25 ml/min with monitoring at 254 nm. The fractions that contained the desired product were concentrated under reduced pressure and the resultant residue was lyophilised from a mixture of dioxane and water. Electrospray MS spectra were obtained on a Micromass platform LCMS spectrometer. Compounds were named using AutoNom 2000.

### EXAMPLE 1

### 1-[2-(2-phenyl-1H-indol-3-yl)ethyl]-N-{([(3,4,5-trimethoxybenzyl)amino]carbonyl}piperidine-4-carboxamide

Step 1: (3,4,5-Trimethoxy-benzyl)-urea

To a solution of 3,4,5-trimethoxybenzylamine (1.0496 g, 5.32 mmol) in water (4 mL) were added conc. HCl (1 mL) and potassium cyanate (3.45 g, 42.5 mmol) and the solution was stirred at 90° C. for 2 h. The mixture was then cooled to room temperature and the solid was filtered and washed with water to yield a white solid (1 g, 78.2%).

¹H-NMR(250MHz, DMSO-d₆) δ=3.60 (s, 3H, —OMe), 3.73 (s, 6H, —OMe), 4.08 (d, 2H, J=6.0 Hz, —CH₂—Ar), 5.54 (s, 2H, —NH₂), 6.42 (t, 1H, J=6.0 Hz, —NH), 6.55 (s, 2H, Harom). HPLC 98%, Rt=1.30 min. MS (ES) m/z 241.24 (M+H).

Step 2: Methanesulfonic acid 2-(2-phenyl-1H-indol-3-yl)-ethyl ester

Methane sulfonyl chloride (0.247 mL, 3.18 mmol) was added dropwise at 0° C. to a solution of 2-(2-phenyl-1H-indol-3-yl)-ethanol (606mg, 2.55mmol) and triethylamine (0.56 mL, 4 mmol) in dry dichloromethane (5 mL). After 40 min the solution was poured into 1N HCl, the organic layer was separated, washed with water, brine, dried over magnesium sulfate and concentrated under vacuum to afford a red oil (0.8 g, 100%).

¹H-NMR(250 MHz, CDCl₃) δ=2.79 (s, 3H, -Me), 3.37 (t, 2H, J=7.4 Hz, —CH₂—Ar), 4.48 (t, 2H, J=7.3 Hz,—CH₂—O—), 7.17-7.34 (m, 4H, Harom), 7.39-7.67 (m, 5H, Harom), 8.20 (s, 1H, —NH). HPLC 94%, Rt=2.95 min. MS (AP) m/z no molecular ion found.

Step 3: 1-[2-(2-Phenyl-1H-indol-3yl)-ethyl]-piperidine-4-carboxylic acid methyl ester

A solution of methanesulfonic acid 2-(2-phenyl-1H-indol-3-yl)-ethyl ester (0.8 g, 2.5 mmol), methyl isonipecotate (0.473 mL, 3.5 mmol) and sodium hydrogen carbonate (0.9 g, 11 mmol) in dry acetonitrile (5 mL) was stirred at 80° C. for 20 h. The solution was cooled, filtered and concentrated under vacuum to afford a yellow oil (0.5 g) that could not be purified by column chromatography due to decomposition on silica or on alumina. The intermediate was engaged in the next step (coupling with urea) without further purification.

Step 4: 1-[2-(2-phenyl-1H-indol-3-yl)ethyl]-N-{[(3,4,5-trimethoxybenzyl)amino]carbonyl}piperidine-4-carboxamide

To a solution of 3,4,5-trimethoxybenzylurea (97.2 mg, 0.4 mmol), 1-[2-(2-phenyl-1H-indol-3yl)-ethyl]-piperidine-4-carboxylic acid methyl ester (220 mg, 0.6 mmol) in dimethylacetamide (3 mL) was added sodium methoxide (0.45 mL, 25% wt sol. in MeOH, 2.0 mmol). The reaction was carried out on a rotary evaporator for 1 h to remove any trace of methanol. Water was then added and the compound extracted with ethyl acetate. The compound was purified by preparative-hplc under basic conditions (DEA) to afford a white solid (29.3 mg, 13%).

¹H-NMR (250 MHz, CDCl₃) δ=1.77-1.90 (m, 4H, piperidine), 2.04 (dd, 2H, J=2.75/11.4 Hz, —CH₂—), 2.12-2.25 (m, 1H, —CH—CO), 2.66-2.72 (m, 2H, —CH₂—), 3.06-3.13 (m, 4H, 2-CH₂), 3.82 (s, 3H, —OMe), 3.85 (s, 6H, —OMe), 4.40 (d, 2H, J=5.8 Hz, —Ar), 6.53 (s, 2H, Harom), 7.11-7.25 (m, 2H, Harom), 7.37-7.67 (m, 7H, Harom), 8.05 (d, 2H, J=7.8 Hz, —NH), 8.72 (t, 1H, J=5.56Hz, —NH). HPLC 100%, Rt=3.59 min. MS (ES) m/z 571.02 (M+H).

### EXAMPLE 2

### 1-[2-(2,6-dimethoxyphenoxy)ethyl]-N-({[3-(trifluoromethyl)phenyl]amino}carbonyl)piperidine-4-carboxamide

To a solution of (3-trifluoromethyl-phenyl)-urea (synthesized using a similar procedure to Example 1, Step 1) (0.1699 g, 0.83 mmol) and 1-[2-(2,6-dimethoxy-phenoxy)-ethyl]-piperidine-4-carboxylic acid methyl ester (synthesized using a similar procedure to Example 1, Step 3) (0.2635 g, 0.81 mmol) in dimethylacetamide (2.0 mL) was added sodium methoxide (0.9 mL, 25% in MeOH, 4.0 mmol). The reaction was stirred for 1 h under vacuum on a rotary evaporator at room temperature. Water (10 mL) was added, a white precipitate formed. The solid was filtered and wash with water. The solid was dissolved in ethyl acetate and the solution washed with water. The organic layer was dried (MgSO₄) and the solvent concentrated to about 0.5 mL. White crystals formed. The crystals were filtered and washed with a small amount of ethyl acetate. The product was obtained as white crystals (0.1157 g, 29%).

¹H-NMR (250 MHz, CDCl₃) δ=1.86-1.95 (m, 4H, CH₂), 2.05-2.19 (m, 2H, —CH₂—), 2.33-2.43 (m, 1H, —CH—CO), 2.80 (t, 2H, J=5.9Hz, —CH₂—), 3.14-3.19 (m, 2H, —CH₂N), 3.83 (s, 6H, 2x-OMe), 4.10 (t, 2H, J=5.9 Hz, —CH₂O), 6.57 (d, 2H, J=8.4 Hz, Harom), 6.99 (t, 1H, J=8.4 Hz, Harom), 7.34-7.47 (m, 2H, Harom), 7.60 (brd, 1H, J=8.0 Hz, Harom), 7.98 (s, 1H, Harom), 9.78 (s, 1H, —NH) and 10.92 (s, 1H, —NH). ¹³C-NMR (62.5 MHz, CDCl₃) δ=26.3, 41.9, 51.1, 54.1, 56.1, 68.5, 103.2, 115.0, 121.2, 121.7, 127.6, 135.2, 135.8, 150.4, 151.7 and 175.9. HPLC 99%, Rt=2.25 min. MS (ES) m/z 496 (M+H).

### EXAMPLE 3

### N-[(cyclohexylamino)carbonyl]-1-[2-(2-phenyl-1H-indol-3-yl)ethyl]piperidine-4-carboxamide

To a solution of cyclohexylurea (synthesized using a similar procedure to Example 1, Step 1) (75.1 mg, 0.5 mmol) and 1-[2-(2-phenyl-1H-indol-3yl)-ethyl]-piperidine-4-carboxylic acid methyl ester (synthesized using a similar procedure to Example 1, Step 3) (253.3 mg, 0.75 mmol) in dimethylacetamide (5 mL) was added sodium methoxide (0.7 mL, 25% wt in MeOH, 3.14 mmol). The reaction was carried out on a rotary evaporator for 1 h to remove any trace of methanol. Water was then added and attempts to extract the compound with ethyl acetate or chloroform failed. The aqueous layer was therefore evaporated to dryness, acetonitrile was added, the solution dried over magnesium sulfate and concentrated to give a yellow solid which was purified by preparative-hplc under basic condition (DEA) and afford a white solid (5.8 mg, 3%). HPLC 100%, Rt=3.97 min. MS (ES) m/z 473.00 (M+H).

### GENERAL PROCEDURE A FOR EXAMPLES 4 AND 5 (LIBRARY COMPOUNDS)

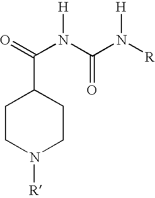

To a stirred solution of urea (synthesized using a similar procedure to Example 1, Step 1) (0.20 mmol) in DMSO (0.5 ml) was added potassium tert-butoxide (0.40 mmol) as a DMSO solution and the reactions shaken at room temperature. After 15 minutes a solution of the ester (synthesized using a similar procedure to Example 1, Step 3) (0.2 mmol) in DMSO was added and the contents shaken for a further 18 hours. The reactions were subsequently filtered over amberlite™-IR-120(H) resin and purified by preparative chromatography using the following conditions:

Mobile phase.

0.2% TFA/water, ACN
Flow rate 25 ml/min.

| | | |
|---|---|---|
| Gradient: | 85/15 H₂0 + 0.2% TFA/ACN | for 1.5 min. |
| | 5/95 in 9.5 min. | for 1.5 min. |
| | 85/15 in 0.5 min. | |

Detector: ELS. (approx. 1.5 ml/min flow split to Sedex 55 ELSD)
Gas (Nitrogen) 2.0 bar
Nebulizer 40° C.
Column: Waters SymmetryPrep ™ 19 mm × 150 mm × 7 μm C18

### EXAMPLE 4

### N-{[(2,4-difluorophenyl)amino]carbonyl}-1-[2-(4-fluorophenyl)ethyl]piperidine-4-carboxamide

Example 4 was synthesized according to General procedure A. HPLC 100%, Rt=3.88 min. MS (AP) m/z 406 (M+H).

### EXAMPLE 5

### N-{[(2-ethoxyphenyl)amino]carbonyl}-1-[2-(4-fluorophenyl)ethyl]piperidine-4-carboxamide

Example 5 was synthesized according to General procedure A. HPLC 97%, Rt =3.96 min. MS (AP) m/z 414 (M+H).

### EXAMPLE 6

Preparation of Tablets

| | |
|---|---|
| Ingredients | mg/tablet |

| | | |
|---|---|---|
| 1. | Active compound of formula (I) | 10.0 |
| | Cellulose, microcrystalline | 57.0 |
| 3. | Calcium hydrogen phosphate | 15.0 |
| 4. | Sodium starch glycolate | 5.0 |
| 5. | Silicon dioxide, colloidal | 0.25 |
| 6. | Magnesium stearate | 0.75 |

The active ingredient 1 is mixed with ingredients 2, 3, 4 and 5 for about 10 minutes. The magnesium stearate is then added, and the resultant mixture is mixed for about 5 minutes and compressed into tablet form with or without fihn-coating.

Primary Screening and IC₅₀ Determination

CHO cells expressing 5-HT_{2A} receptors seeded in 384 well plates are pre-loaded with Fluo-4AM fluorescent dye and then incubated with compound (10 μM for primary screen) for 15 min. Fluorescent intensity is recorded using a Fluorometric imaging plate reader (FLIPR384, Molecular Devices) and inhibition of the peak response evoked by 5-HT (EC₇₀ concentration) is calculated.

IC₅₀ determinations are performed utilizing the same functional assay as described for primary screening (15 min antagonist compound pre-incubation), applying the compounds in the dose range of 3 nM to 10 μM.

In Vitro Receptor Pharmacology Selectivity Determinations

The affinity constants of compounds were determined using recombinant human serotonin receptors stably expressed in fibroblast cell lines (CHO or HEK293), measuring the ability of the compounds to displace radio-labelled tracers using scintillation proximity assays or filter binding assays. For 5-HT_{1B}, 5-HT_{2B} and 5-HT_{2C} receptor binding studies ³H-LSD was used as radio ligand, for 5-HT_{2A} and 5-HT₆ ³H-5-HT was used as tracer, while the binding constant to 5-HT_{1A} was determined using ³H-8-OH-DPAT. The non-selective serotonin receptor antagonist mianserine was used as reference substance.

The activity at 5-HT_{2C} receptors was studied in a FLIPR based assay, measuring the effect of compounds on 10 nM 5-HT induced_Ca²⁺-currents.

The calculation of the Kᵢ values for the inhibitors was performed by use of Activity Base. The Kᵢ value is calculated from IC₅₀ using the Cheng Prushoff equation (with reversible inhibition that follows the Michaelis-Menten equation): Kᵢ=IC₅₀ (1+[S]/Kₘ) [Cheng, Y. C.; Prushoff, W. H. Biochem. Pharmacol. 1973, 22, 3099-3108]. The compounds of Formula (I) exhibit IC₅₀ values for the 5-HT_{2A} receptor in the range from 1 nM to 10 μM.

5-HT_{2A} antagonist lead compounds were identified in FLIPR-based functional screening of the 5-HT_{2A} receptor. One of these compounds were tested in equilibrium displacement binding measurements. The results show that Example 2 is a high affinity ligand for the 5-HT_{2A} receptor subtype, with a Kᵢ value in the nanomolar range. The compound is highly selective over five other serotonin receptors assayed (5-HT_{2C}, 5-HT_{2B}, 5-HT_{1A}, 5-HT₆ and 5-HT_{1B}). Example 2 is shown also to be selective at 5-HT_{2A} versus the 5-HT_{2C} receptor in terms of efficacy.

| | Functional | | | | | | |
|---|---|---|---|---|---|---|---|
| | Kᵢ(nM) | Binding Kᵢ(nM) | | | | | |
| Example | 5-HT_{2A} | 5-HT_{1A} | 5-HT_{1B} | 5-HT_{2A} | 5-HT_{2B} | 5-HT_{2C} | 5-HT₆ |
| Example 2 | 32.3 | >1000 | >1000 | 23 | >1000 | >1000 | >1000 |

The table shows the selectivity of Example 2 for the 5-HT_{2A} over other serotonin-binding receptors.

A number of embodiments of the invention have been described. Nevertheless, it will be understood that various modifications may be made without departing from the spirit and scope of the invention. Accordingly, other embodiments are within the scope of the following claims.

1. A compound of the Formula (I) 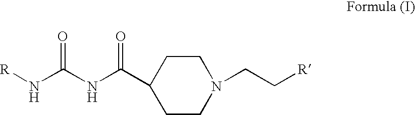
wherein R is selected from: aryl optionally independently substituted with one or more of C₁₋₆-alkyl, C₁₋₆-alkoxy, halogen, and halo-C₁₋₆-alkyl; aryl-C₁₋₆-alkyl optionally independently substituted with one or more of C₁₋₆-alkoxy; and C₃₋₈-cycloalkyl;
R′ is selected from aryl optionally independently substituted with one or more of halogen, C₁₋₆-alkoxy, halo-C₁₋₆-alkyl, and cyano; aryloxy optionally independently substituted with one or more of halogen and C₁₋₆-alkoxy; heteroaryl optionally independently substituted with one aryl and/or one or more of halogen, C₁₋₆-alkyl, and C₁₋₆-alkoxy, which aryl is optionally independently substituted with one or more of halogen, C₁₋₆-alkyl, and C₁₋₆-alkoxy;
and pharmaceutically acceptable salts, hydrates, solvates, geometrical isomers, tautomers, optical isomers, and prodrug forms thereof:

2. A compound according to claim 1, wherein R is selected from: phenyl independently substituted with one or more of methyl, methoxy, ethoxy, fluoro, and trifluoromethyl; benzyl independently substituted with one or more of methoxy; and cyclohexyl.

3. A compound according to claim 1, wherein R is selected from 2-ethoxyphenyl, 2,4-difluorophenyl, 3-(trifluoromethyl)phenyl, 3,4,5-trimethoxybenzyl, and cyclohexyl.

4. A compound according to claim 1, wherein R′ is selected from: phenyl independently substituted with one or more of fluoro; phenoxy independently substituted with one or more of methoxy; and indolyl independently substituted with one phenyl and/or one or more of fluoro, chloro, methyl, and methoxy, which phenyl is optionally independently substituted with one or more of fluoro, chloro, methyl, and methoxy.

5. A compound according to claim 1, wherein R′ is selected from 4-fluorophenyl, 2,6-dimethoxyphenoxy, and 2-phenyl-3-indolyl.

6. A compound according to claim 1, which is selected from:
1-[2-(2-phenyl-1H-indol-3-yl)ethyl]-N-{[(3,4,5-trimethoxybenzyl)amino]carbonyl}piperidine-4-carboxamide,
1-[2-(2,6-dimethoxyphenoxy)ethyl]-N-( {[3-(trifluoromethyl)phenyl]amino}carbonyl)piperidine-4-carboxamide,
N-[(cyclohexylamino)carbonyl]-1-[2-(2-phenyl-1H-indol-3-yl)ethyl]piperidine-4-carboxamide,
N-{[(2,4-difluorophenyl)amino]carbonyl}-1-[2-(4-fluorophenyl)ethyl]piperidine-4-carboxamide, and
N-{[(2-ethoxyphenyl)amino]carbonyl}-1-[2-(4-fluorophenyl)ethyl]piperidine-4-carboxamide.

7. A process for the preparation of a compound according to claim 1, which process comprises the steps of:
a) reacting an amine RNH₂
wherein R is: aryl optionally independently substituted with one or more of C₁₋₆-alkyl, C₁₋₆-alkoxy, halogen, and halo-C₁₋₆-alkyl; aryl-C₁₋₆-alkyl optionally independently substituted with one or more of C₁₋₆-alkoxy; C₃₋₈-cycloalkyl;
with a cyanate, to give a compound of formula R—NH—CO—NH₂,
wherein R is as defined above;
b) alkylation of a compound of Formula (II) 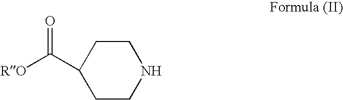
wherein R″ is C₁₋₆-alkyl,
via displacement of a leaving group by reaction of the compound of Formula (II) with an alkylating agent of the Formula R′—CH₂—CH₂-LG,
wherein R′ is: aryl optionally independently substituted with one or more of halogen, C₁₋₆-alkoxy, halo-C₁₋₆-alkyl, and cyano; aryloxy optionally independently substituted with one or more of halogen and C₁₋₆-alkoxy; heteroaryl optionally independently substituted with one aryl and/or one or more of halogen, C₁₋₆-alkyl, and C₁₋₆-alkoxy, which aryl is optionally independently substituted with one or more of halogen, C₁₋₆-alkyl, and C₁₋₆-alkoxy; and
LG is a leaving group,
to give a compound of Formula (III) 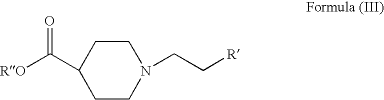
wherein R′ and R″ are as defined above,
c) reacting the products from steps a) and b) in the presence of a base, such as sodium methoxide or potassium tert-butoxide, to give a compound of Formula (I) 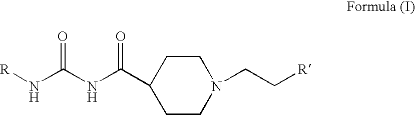
wherein R and R′ are as defined above.

8. A pharmaceutical formulation comprising a compound according to claim 1 as active ingredient, in combination with a pharmaceutically acceptable diluent or carrier.

9. A method for the prophylaxis or treatment of a 5-HT_{2A} receptor-related disorder, which comprises administering to a subject in need of such treatment an effective amount of a compound according to claim 1.

10. The method according to claim 9, wherein the disorder is selected from schizophrenia, mental depression, migraine, epilepsy, obsessive-compulsive disorder, sleep disorders such as insomnia and obstructive sleep apnea, anorexia nervosa, cardiovascular conditions such as hypertension, vasospasm, angina, Raynaud's phenomenon and thrombotic illness including stroke, glaucoma, alcohol and cocaine dependence.

11. A method for modulating 5-HT_{2A} receptor activity, which comprises administering to a subject in need of such treatment an effective amount of a compound according to claim 1.
